(19) United States
(12) Patent Application Publication
(10) Pub. No.: US 2025/0026074 A1
(43) Pub. Date: Jan. 23, 2025

(54) 3D-PRINTING ENGINEERED LIVING MATERIALS

(71) Applicant: The Trustees of Princeton University, Princeton, NJ (US)

(72) Inventors: Sujit S. DATTA, Princeton, NJ (US); Rodney PRIESTLEY, Princeton, NJ (US); Xiaohui XU, Princeton, NJ (US); Reed Konane BAY, Princeton, NJ (US)

(73) Assignee: The Trustees of Princeton University, Princeton, NJ (US)

(21) Appl. No.: 18/707,400

(22) PCT Filed: Nov. 4, 2022
(86) PCT No.: PCT/US2022/048912

§ 371 Date: May 3, 2024

Related U.S. Application Data
(60) Provisional application No. 63/275,565, filed on Nov. 4, 2021.

(51) Int. Cl. B29C 64/106; B29C 64/209; B29C 64/264; B29C 64/40; B29K 33/00; B29K 105/00; B33Y 10/00; B33Y 30/00; C12N 1/20

(52) U.S. Cl. CPC B29C 64/106; B29C 64/209; B29C 64/264; B29C 64/40; B33Y 10/00; B33Y 30/00; C12N 1/20; B29K 2033/26; B29K 2105/0005

(57) ABSTRACT

Disclosed is a method to 3D print materials with defined bacterial communities into controlled, complex 3D structures, and compositions. The technique includes first providing an ink composition that includes a pre-polymer composition and a microorganism, where the pre-polymer composition includes a polymerizable monomer, a cross-linking agent, the photoinitiator, and a solvent. The technique also includes 3D printing a pattern in a hydrogel support matrix using the ink composition where the hydrogel support matrix is in a container. The technique may also include forming a 3D printed engineered living material by curing the 3D printed pattern.

### CROSS-REFERENCE TO RELATED APPLICATIONS

The present application claims priority to U.S. Provisional Patent App. No. 63/275,565, filed Nov. 4, 2021, the contents of which are incorporated by reference herein in their entirety.

### TECHNICAL FIELD

The present disclosure is drawn to systems and method for controlling bacterial growth, and specifically, to 3D printing of bacterial colonies.

### BACKGROUND

This section is intended to introduce the reader to various aspects of art, which may be related to various aspects of the present invention that are described and/or claimed below. This discussion is believed to be helpful in providing the reader with background information to facilitate a better understanding of the various aspects of the present invention. Accordingly, it should be understood that these statements are to be read in this light, and not as admissions of prior art.

Bacteria are known to thrive in diverse ecosystems and habitats. In nature, bacteria can be found growing on surfaces, which in addition to the ease of visualization in two dimensions, have led laboratory studies to typically focus on colony growth on two-dimensional (2D) planar surfaces. However, in many cases in nature, bacteria grow in three-dimensional (3D) habitats, such as gels and tissues inside of hosts, soils and other subsurface media, wastewater treatment devices, and naturally occurring bodies of water. Nonetheless, despite their prevalence, the morphodynamics of bacterial colonies growing in such 3D environments remains largely unknown.

Therefore, a means and system for providing engineered bacterial colonies in three dimensions is desirable.

### BRIEF SUMMARY

Various deficiencies in the prior art are addressed below by the disclosed system and method.

A method for 3D printing engineered living materials may be provided. The method may include providing an ink composition, 3D printing a pattern in a hydrogel support matrix using the ink composition, the hydrogel support matrix being in a container, and forming a 3D printed engineered living material by curing the 3D printed pattern. The ink composition may include a pre-polymer composition and a microorganism, the pre-polymer composition comprising a polymerizable monomer (such as acrylamide), a cross-linking agent (such as N,N′-methylenebis(acrylamide)), the photoinitiator (such as 2-Hydroxy-4′-(2-hydroxyethoxy)-2-methylpropiophenone), and a solvent.

While there is no restriction on the microorganisms that may be incorporated, in some embodiments, the microorganism may be, e.g., a species of E. coli, A. adeninivorans, S. cerevisiae, or C. glutamicum. In some embodiments, more that one species may be incorporated. In some embodiments, the

In some embodiments, the method may advantageously include providing the ink composition by first forming an ink composition by mixing a pellet having a known cell amount with a pre-polymer composition, then centrifuging the ink composition, and then loading the centrifuged ink composition into a container for injection, which may be, e.g., a syringe.

In some embodiments, the method may advantageously include forming the pellet by first inoculating cells of the microorganism into a growth medium, then growing the inoculated cells in the growth medium, and then centrifuging the grown cells into a pellet.

In some embodiments, the method may advantageously include forming the pre-polymer composition by mixing the polymerizable monomer, the cross-linking agent, the photoinitiator, and the solvent. In some embodiments, forming the pre-polymer composition may include incorporating (e.g., mixing) a thickening agent (such as sodium alginate) into the pre-polymer composition.

In some embodiments, the method may advantageously include forming the hydrogel support matrix by first forming a hydrogel composition by mixing a sterile growth medium with a thickening agent, then eliminating bubbles by centrifuging the hydrogel composition, and then loading the hydrogel composition into a container for printing into.

In some embodiments, curing the 3D printed pattern may include irradiating the 3D printed pattern with at least one wavelength of light configured to activate the photoinitiator.

The method may also advantageously include reducing the viscosity of the hydrogel matrix to allow the 3D printed engineered living material to be released from the matrix, for example, by contacting at least part of the hydrogel matrix with a buffered solution such as a 10× phosphate buffered solution (PBS).

A system for 3D printing engineered living materials may also be provided. Such as system may include a container that includes a hydrogel support matrix as disclosed herein. The system may include a reservoir containing an ink, the ink including a pre-polymer composition and a microorganism, the pre-polymer composition comprising a polymerizable monomer, a crosslinking agent, the photoinitiator, and a solvent as disclosed herein. The system may include a print head configured to deposit ink from the reservoir into the hydrogel support matrix, which may include, e.g., an injection needle, which may have a gauge between 15 and 34. The system may include a light source configured to irradiate the deposited ink with at least one wavelength of light capable of activating the photoinitiator. The system may include at least one processor operably coupled to, and configured to control, the print head and light source.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a flowchart of an embodiments of a method.

FIG. 2 is a block diagram of an embodiment of a system.

FIG. 3 is a schematic diagram of an example system.

FIGS. 4A-4D are images of a 3D printed letter “P” in a hydrogel support bath, dyed pink for ease of imaging, at time t=0 (prior to curing) (4A), after curing (4B), after 24 hours (4C) and after 48 hours (4D).

FIG. 5 is a schematic illustration of an example printed structure.

### DETAILED DESCRIPTION

From a physics standpoint, growing in three dimensions is fundamentally different from growing in two dimensions in terms of both nutrient access and the ability to grow and deform into an additional dimension. Consequently, we expect colony morphodynamics—the way a colony's overall shape changes over time—to also be different. Some recent studies hint that this is indeed the case, showing how specific mechanical interactions imposed by a 3D environment can influence the morphology of growing biofilms. For instance, external fluid flows are now known to trigger the formation of streamers that stem from an initially surface-attached colony. Also, under quiescent conditions, small (at most tens of cells across) biofilm colonies constrained in cross-linked gels adopt internally ordered structures as they grow and push outward, mediated by elastic stresses arising at the interface between the colony and its stiff environment. However, the behavior of larger bacterial colonies growing freely in quiescent 3D environments remains underexplored, despite the fact that they represent a fundamental building block of more complex natural colonies.

The present disclosed system and method allow one to consider questions such as: what determines the shape of a bacterial colony growing in three dimensions? And are there general characteristics and universal principles that span across species and specific environmental conditions?

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments, such as seismology and data fusion.

Various embodiments are directed to a method and system to 3D print engineered living materials.

Previous technologies for 3D-printing of engineered living materials composed of bacteria housed in a polymeric matrix mixed the bacteria and pre-polymers together and 3D printed onto flat surfaces. However, the bacteria and pre-polymers are often very soft, so they will slump when printed on a surface, which hinders the ability to print complex shapes.

Broadly, the 3D printing technique disclosed here has dense bacteria colonies printed in a bed of hydrogel matrix support. Here, the hydrogel support is used to print complex structures of bacteria embedded in pre-polymer. In the support bath, the bacteria and pre-polymer are then cured with UV, which fixes the bacteria in place. It was heretofore unknown to print bacteria and pre-polymer into a hydrogel support bath to create 3D printed biohybrid materials.

An advantage of the hydrogel support bath is the ability to introduce the nutrient for the bacteria to grow and theoretical one can control the gradient of that nutrient within the hydrogel matrix support. Through controlling the nutrient gradient, one can direct the bacteria to migrate through the pre-polymer towards high nutrient regions and set-up a gradient of bacteria within the printed material before curing pre-polymer in UV. Regions of higher concentrations of nutrient/bacteria will have different properties then the regions with less. This sets up the potential for stimuli-responsive materials.

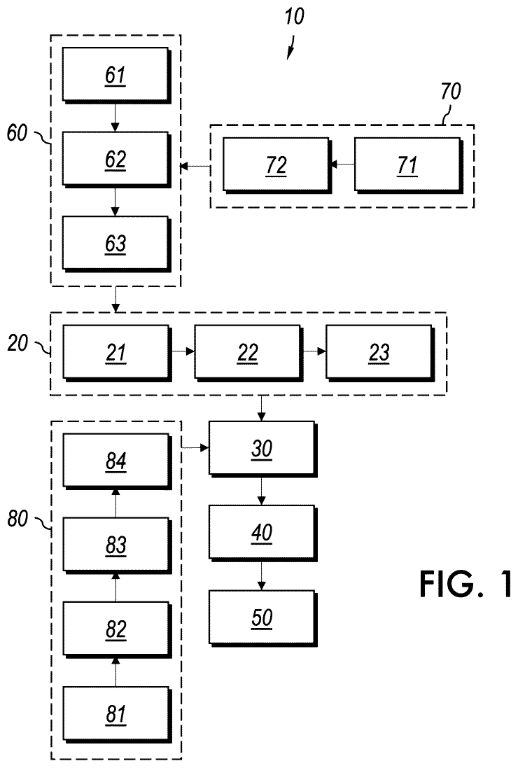

Referring to FIG. 1, a method 10 for 3D printing engineered living materials may be provided. The method may include providing 20 an ink composition. The ink composition may include a pre-polymer composition and a microorganism.

### a. Pre-Polymer Composition

The pre-polymer composition will generally include a polymerizable monomer, a cross-linking agent, the photoinitiator, and a solvent. In some embodiments, the pre-polymer composition may include a thickening agent.

The polymerizable monomer may be any appropriate monomer. Non-limiting examples include lactic acid, glycolic acid, acrylic acid, 1-hydroxyethyl methacrylate, ethyl methacrylate, 2-hydroxyethyl methacrylate (HEMA), propylene glycol methacrylate, acrylamide, N-vinylpyrrolidone (NVP), methyl methacrylate, glycidyl methacrylate, glycerol methacrylate (GMA), or glycol methacrylate. In a preferred embodiment, the polymerizable monomer is acrylamide.

The cross-linking agent may be any appropriate cross-linking agent. In some embodiments, bifunctional or polyfunctional acrulic monomers, such as (meth)acrylamide having two or more polymerizable carbon-carbon double bonds in a molecule can be used. Non-limining examples include N,N′-methylenebisacrylamide, N,N′methylene bismethacrylamide, N,N′-ethylene bisacrylamide, N,N′-ethylene bismethacrylamide, N,N′propylenebisacrylamide and N,N′-(1,2-dihydroxyethylene)bisacrylamide. In a preferred embodiment, the crosslinking agent is N,N′-methylenebis (acrylamide).

The photoinitiator may be any appropriate photoinitiator that is soluble in the solvent. Such photoinitiators may include, e.g., benzoin derivatives, methylolbenzoin and 4-benzoyl-1,3-dioxolane derivatives, benzilketals, α,α-dialkoxyacetophenones, α-hydroxy alkylphenones, α-aminoalkylphenones, acylphosphine oxides, bisacylphosphine oxides, acylphosphine sulphides, and halogenated acetophenone derivatives, and the like. Non-limiting examples of such photoinitiators include 4-[2-(4-morpholino)benzoyl-2-dimethylamino]-butylbenzenesulfonate salt, and phenyl-2,4,6-trimethyl-benzoylphosphinate salt (suitable salts include, for example, sodium and lithium cations). In a preferred embodiment, the photoinitiator is 2-hydroxy-4′-(2-hydroxyethoxy)-2-methyl propiophenone.

The solvent may be any appropriate solvent that is not toxic to the microorganism(s), but will typically comprise or consist of water.

The thickening agent may be any appropriate thickening agent. In some embodiments, the thickening agent is a biopolymeric thickening agent. In some embodiments, the biopolymeric thickening agent may include a sugar polymer. Sugar polymers include, for example, agar, sodium alginate, celluloses (such as carboxymethylcellulose), pectin and carrageenan. In a preferred embodiment, the thickening agent is sodium alginate.

Referring to FIG. 1, the method may include forming 70 the pre-polymer composition by mixing 71 the polymerizable monomer, the cross-linking agent, the photoinitiator, and the solvent. In some embodiments, this may also include mixing 72 a thickening agent into the pre-polymer composition.

### a. Microorganism

The disclosed approach is agnostic with respect to microorganisms that can be utilized here. The microorganism is preferably a species of bacteria. In some embodiments, a single species is utilized. In some embodiments, a plurality of species are utilized. In some embodiments, the microorganism may include a prokaryotic and/or eukaryotic species. Some such species are listed below. However, it will be appreciated that other species can be suitable.

Non-limiting examples of microorganisms includes species from a genus such as Aspergillus, Blastobotrys/Arxula, Candida, Corynebacterium, Escherichia, Lactobacillus, Laetiporus, Lentinus, Phanerochaete, Pichia, Physcomitrella, Rhodoturula, Saccharomyces, Schizosaccharomyces, Sphaceloma, Xanthophyllomyces or Yarrowia. Non-limiting examples of species of such genera include A. flavus, A. fumigatus, A. niger, A. terreus, A. adeninivorans, C. albicans, C. glabrata, C. tropicalis, C. utilis, C. glutamicum, C. pseudotuberculosis, C. xerosis, E. coli, L. acidophilus, L. crispatus, L. helveticus, L. jensenii, L. sulphureus, L. tigrinus, P. chrysosporium, P. pastoris, P. patens, R. glutinis, R. mucilaginosa, S. cerevisiae, S. pombe, S. arachidis, and Y. lipolytica. In a preferred embodiment, the microorganism is a species of E. coli, A. adeninivorans, S. cerevisiae, C. glutamicum, or a combination thereof.

Referring to FIG. 1, providing 20 the ink composition may include forming 21 an ink composition by mixing a pellet having a known cell amount with a pre-polymer composition, then centrifuging 22 the ink composition, and then loading 23 the centrifuged ink composition into a container for injection.

The container for injection may be any appropriate container that can be used to inject the ink into a hydrogel. It may be a container adapted or configured for use with a 3D printer. It may be, e.g., a syringe. The injection may use a needle or microcapillary. In some embodiments, the needle may have a gauge between 15 and 34.

In some embodiments, the pellet may be provided, or may be formed 60 by inoculating 61 cells of the microorganism into a growth medium, growing 62 the inoculated cells in the growth medium, and centrifuging 63 the grown cells into a pellet.

Referring to FIG. 1, after the ink composition has been provided, the method may include 3D printing 30 a pattern in a hydrogel support matrix using the ink composition. The hydrogel support matrix may be in a container. The hydrogel is preferably an optically transparent or translucent hydrogel.

In some embodiments, the method may include forming 80 a hydrogel support matrix. This may include forming 81 a hydrogel composition by mixing a sterile growth medium with a thickening agent. Optionally, this may include adjusting 82 the pH of the hydrogel. This may include eliminating 83 bubbles by centrifuging the hydrogel composition. This may also include loading 84 the hydrogel composition into a container for printing into. The container is preferably optically transparent, such as glass.

The purpose of the hydrogel is, in part, to restrict the growth of the bacterial colonies that will be printed within the hydrogel matrix. The hydrogel matrices preferably have four notable characteristics: (I) they are transparent, enabling us to directly visualize colony morphodynamics in situ; (II) They are easily mechanically deformed and rearranged (yielded), and thus, they do not strongly constrain colony growth but simply keep the cells suspended in 3D; (III) They can be designed to be replete with oxygen and nutrients given that the internal mesh size of the individual hydrogel grains is ˜40 to 100 nm and thus, permissive of free diffusion of small molecules throughout each matrix, thereby sustaining cellular proliferation over many generations; and (IV) The sizes of the interstitial pores between adjacent hydrogel grains can be precisely tuned by changing the hydrogel grain packing density.

As an example, Escherichia coli colonies have long cylindrical shapes with radii R that vary between ˜20 and 250 μm, at least tens of cells across. To isolate the influence of cellular growth on colony morphodynamics, hydrogel matrices with mean pore sizes between ˜0.1 and 1.0 μm (i.e., smaller or comparable with the diameter of a single cell) may be used. Hence, the bacteria are stuck inside the pores; even if they are nominally motile, they cannot self-propel through the pore space. Nevertheless, as the cells consume nutrients, they grow—transiently deforming and yielding the surrounding matrix—and push their progeny out into the neighboring available pores.

Generally, the swollen hydrogel granules, which may be ˜5 to 10 μm diameter, pack tightly against each other, resulting in a jammed matrix; the individual hydrogel granules have an internal mesh size of ˜40 to 100 nm. Thus, small molecules (e.g., amino acids, glucose, oxygen) can freely diffuse throughout the medium, while the cells themselves are confined in the interstitial pore space between hydrogel granules.

The hydrogel will typically comprise at least two components—(1) a liquid component, and (2) a gelling agent capable of interacting with the liquid component. In some embodiments, the hydrogel may be formed by swelling the gelling agent (e.g., dry copolymer particles) in an appropriate liquid cell culture media.

The liquid component is preferably a liquid cell culture media selected as appropriate for the microorganism(s) in the ink. For example, EZ Rich (Teknova Inc.), a defined rich medium, was used for experiments with E. coli; 2% Lennox LB (Sigma Aldrich) for the experiments V. cholerae and P. aeruginosa; and Mannitol-Yeast Extract-Peptone (MYP) medium (Sigma Aldrich) for experiments with K. sucrofermentans. The components to prepare the EZ Rich were mixed following manufacturer directions; specifically, the liquid medium is an aqueous solution of 10× MOPS Mixture (M2101), 10× ACGU solution (M2103), 5× Supplement EZ solution (M2104), 20% glucose solution (G0520), 0.132 M potassium phosphate dibasic solution (M2102), and ultrapure milli-Q water at volume fractions of 10%, 10%, 20%, 1%, 1%, and 58%, respectively. All components except glucose are combined and then autoclaved. After the medium cools, sterile glucose is added. The MYP medium is made of D-Mannitol (25 g/L), Yeast Extract (5 g/L), and Peptone (3 g/L) all mixed in ultrapure milli-Q water and autoclaved.

Any appropriate gelling agent capable of forming a hydrogel in the liquid cell culture media may be used. For example, in some embodiments, the hydrogel is formed using a crosslinked polyacrylic acid. Preferably the polyacrylic acid is a homopolymer of acrylic acid crosslinked with an allyl ether of pentaerythritol, allyl ether sucrose, propylene, polyalkelyether or divinyl glycol. Acrylates/C10-30 alkyl acrylate crosspolymers are copolymers of C10-30 alkyl acrylates and one or more monomers of acrylic acid, methacrylic acid or their simple esters thereof crosslinked with an allyl ether of sucrose or pentaerythritol. The designation “C10-30” refers to 10 to 30 carbon atoms. In preferred embodiments, the crosslinked polyacrylic acid is an acrylic acid/alkyl acrylate copolymer.

In some embodiments, a high molecular weight polymer of a crosslinked polyacrylic acid sold under the trade name “Carbopol” is available from Lubrizol or Ashland. Carbopol polymers are typically pH sensitive; at acidic pH the polymer is uncharged whereas at neutral pH the acid groups are deprotonated and result in a negatively charged polymer. The anionic form of the polymer has the ability to absorb and retain water and swell to many times of their original volume thereby forming a hydrogel. Non-limiting examples of gelling agents include Carbopol 934, 934P, 940, 941, 954, 980, 981, 1342, 1382, 2984, and 5984; Aqua SF-1 polymer; and Carbopol ETD 2001 and ETD 2050; and Carbopol Ultrez 10. In a most preferred embodiment, Carbopol 980 is utilized. Acrylates/C10-30 alkyl acrylate crosspolymers are also available from Noveon Incorporated under the tradename Pemulen TRI or TR2, Carbopol 1342 or 1382, Carbopol ETD 2020, and Carbopol Ultrez 20 and 21.

The hydrogel granules may be homogeneously dispersed by mixing each dispersion for a high shear rate for a short period of time, or a low shear rate for a longer period of time. For example, in some embodiments, the two components are mix for at least 2 h at 1600 rpm using magnetic stirring or in a stand mixer (Hamilton Beach 730C Classic DrinkMaster Mixer) for 2 minutes.

The pH may also be adjusted as appropriate. This is preferably done after the gelling agent has been mixed into the liquid. In some embodiments, the pH may be adjusted to a target pH to ensure cell viability. Any appropriate pH adjusting agent may be used. Often, especially with acidic gelling agents that must be neutralized, such pH adjusting agents are bases, such as KOH or NaOH. For example, in some embodiments, pH was adjusted to 7.4 by adding 10 M NaOH to ensure cell viability.

The hydrogel may contain other components as appropriate for the intended purpose. For example, in some embodiments, the hydrogel may include one or more chemical agents, to determine if the chemical agent(s) have an effect on the microorganism growth. In some embodiments, the hydrogel may include one or more colorants (e.g., dyes, etc.) for improved imaging or other diagnostic purpose. In some embodiments, the hydrogel may contain one or more precursor materials, that the microorganism may use for production of various metabolites, including, e.g., industrial chemicals.

Referring to FIG. 1, the method may include forming 40 a 3D printed engineered living material by curing the 3D printed pattern. This may be done in any appropriate manner, based on the photoinitiator chosen, provided the means for activating the photoinitiator does not also kill the microorganism. In some embodiments, this may include irradiating the 3D printed pattern with at least one wavelength of light configured to activate the photoinitiator. Preferably, the photoinitiation step will be configured to have a minimal impact on the microorganism—for example, UV curing of the polymer could also lead the microorganism to mutate and some cell death.

Referring to FIG. 1, the method may include releasing 50 the printed pattern from the support matrix. This may include reducing the viscosity of the hydrogel matrix to allow the 3D printed engineered living material to be released from the matrix. Reducing the viscosity may be accomplished by, e.g., contacting at least part the hydrogel matrix with a buffered solution. The buffered solution may be, e.g., a 10× phosphate buffered solution.

However, cell viability may potentially be reduced if removed from hydrogel support matrix. The cells can potentially be overcome by re-awakening the cells through placing the print back in liquid media.

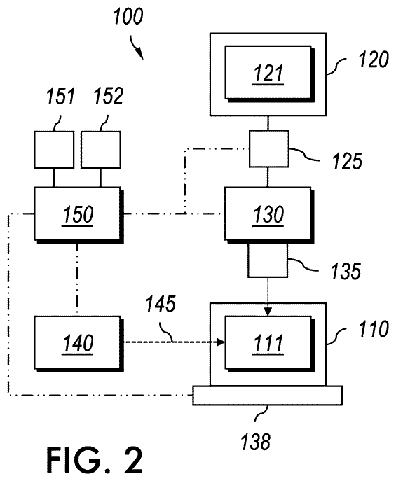

In some embodiments, a system for 3D printing engineered living materials may be provided. Referring to FIG. 2, the system 100 may include a container 110 as disclosed herein comprising a hydrogel support matrix 111 as disclosed herein. The system may include a reservoir 120 containing an ink 121. The ink composition, as disclosed herein, may include a pre-polymer composition and a microorganism, where the pre-polymer composition may include a polymerizable monomer, a crosslinking agent, the photoinitiator, and a solvent. The system may include a print head 130 configured to deposit the ink from the reservoir into the hydrogel support matrix. In some embodiments, the print head may include an injection needle 135. The injection needle may have a gauge between 15 and 24. The system may include a light source 140 configured to irradiate the deposited ink in the hydrogel support matrix with at least one wavelength of light 145 capable of activating the photoinitiator. The system may include at least one processor 150 operably coupled to the light source and the print head.

In some embodiments, the processor may be operably coupled to a mobile stage 138 (upon which the container 110 is placed). In some embodiments, the processor may be coupled to the print head and the mobile stage. In some embodiments, the processor may be coupled to the print head or the mobile stage.

In some embodiments, the processor may be operably coupled to a pump 125 or piston. The pump may be configured to cause ink to flow from the reservoir to the print head. The pump is shown as being between the reservoir and the print head, but other configurations may be utilized as appropriate. For example, in some embodiments (not shown), the ink may be contained in a syringe, where the piston pushes down on the ink to force the ink to flow to the print head.

The processor may be coupled to a memory 151 and a non-transitory computer readable storage medium 152. The processor, memory, and non-transitory computer readable storage medium may be a part of, e.g., a desktop or laptop computer. The non-transitory computer readable storage medium may contain instructions that, when executed, configure the at least one processor to control the print head and/or light source. In some embodiments, the instructions may configure the processor to receive a desired print pattern, determine an appropriate path to form the desired print pattern, optionally receive input regarding a user's desired print settings, cause the print head to deposit the ink according to the determined path. In some embodiments, the instructions may configure the processor, in addition to or alternatively to the above, to activate the light source at a predetermined dose for a predetermined dosing schedule.

To explore the morphodynamics of dense microorganism colonies growing in 3D environments, the bioprinter will typically inject densely packed (e.g., number density ρ˜10¹⁰ to ρ˜10¹⁴ cells/mL, and preferably ρ˜10¹²) colonies of a microorganism inside granular hydrogel matrices.

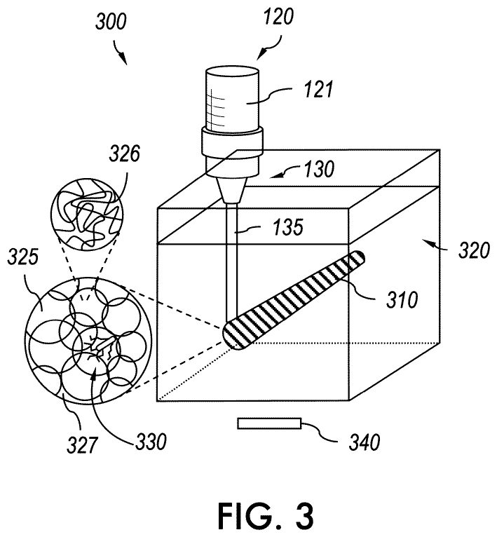

A simplified illustration of the printing step can be seen in FIG. 3. FIG. 3 shows a schematic of experiments in using a system 300 which a dense bacterial colony 310 (here, shown as a simple cylindrical shape) is 3D printed within a transparent matrix 320 made of jammed hydrogel grains 325, each grain comprising the polymer 326 used to form the hydrogel. The matrix locally yields as the ink (including the cells) are injected into the pore space, and then, it rapidly rejams around the dense-packed cells, holding them in place. The individual hydrogel grains are highly swollen in liquid media 327 containing, e.g., salts and nutrients, which can freely pass through. However, the interstitial pores between grains are smaller than the body size of the cell 330, thereby suppressing any motility and holding any cells in place. The colony then expands outward solely through cellular growth and division into adjacent available pores. This generally extends outwards from the printed ink pattern. Various detection and visualization systems can be used to observe the printed cells, which may include used of, e.g., an objective lens 340. For example, confocal microscopy can be used to obtain 3D stacks of optical slices of cell body fluorescence at different depths in the medium.

### Example 1

As a simplified example of the disclosed method, a pre-polymer composition was first formed by dissolving 0.35 g acrylamide, 20 mg Irgacure® 2959 and 1.5 mg N,N′-methylenebis(acrylamide) in 0.5 mL DI water, and then adding 2 ml 4 wt % sodium alginate and mixing until uniform.

To form a pellet, E. coli D1 cells were inoculated from a stock agar plate into 2 mL of Luria-Bertani broth (LB). The cells were grown in 150 rpm shaking conditions at 37 C overnight. 100 μL of the overnight cells were inoculated into 2 mL of fresh LB. Cells were grown for 2 hours until the OD was ˜0.5. 1 mL was transferred into Eppendorf tubes, which were used to centrifuge (4000 rpm for 8 minutes) into a pellet.

The ink composition was created by mixing the pellet (with the known cell amount) with 1 mL of the pre-polymer composition disclosed previously. An optional water-soluble dye was also added to the composition as an aid in imaging. A syringe was used to mix the combination. This combination was then centrifuged at 4000 rpm for approximately 30 seconds to remove bubbles, after which the combination was loaded into a syringe with a 20-gauge needle for printing.

Separately, a milkshake blender was used to mix sterile LB with Carbopol to yield a 1% Carbopol gel. Once mixed, the pH of the gel was determined, and adjusted until a desired pH was achieved. Here, a 10 M NaOH solution was added until a pH of 7.4 was reached. The resulting support matrix was optionally placed in a 15 mL centrifuge tube, where it was centrifuged to eliminate all, or substantially all, of the bubbles. The hydrogel support matrix was then loaded into a printing container (here, a borosilicate glass beaker).

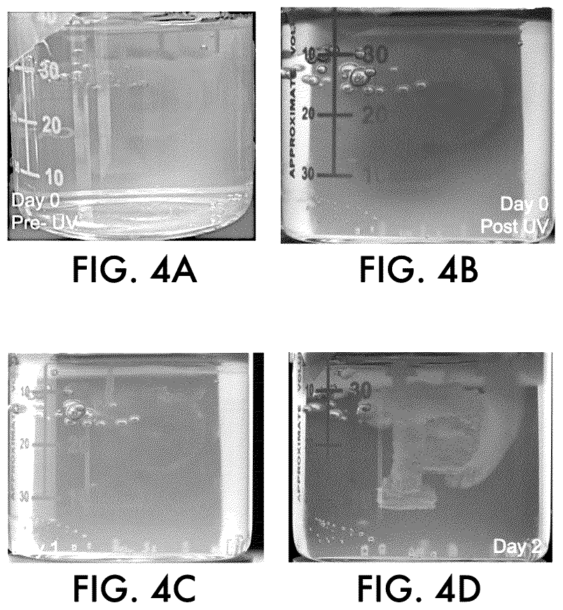

To 3D print the desired shape, the syringe containing the ink composition was loaded into a bioprinter, here a modified Lulzbot® 3D printer. A design file containing a desired print pattern was loaded into software configured to control the bioprinter, and the desired print settings (print speed, layer thickness, travel speed, etc) were selected. The bioprinter then printed the desired print pattern into the borosilicate glass beaker and specifically into the hydrogel support matrix in the borosilicate glass beaker. An image of the resulting printed pattern can be seen in FIG. 4A.

The pattern was then cured by taking the resulting printed pattern and hydrogel matrix, and irradiating it with a UVGL-58 handheld UV lamps (1 mW/cm2, λ=365 nm) for 60 min. The resulting cured pattern is seen in FIG. 4B.

The printed material was then grown for 2 days. After the first day, an image was captured (see FIG. 4C), and after 2 days, a second image was collected (see FIG. 4D). As seen, after two days, the E. coli colonies are visible, and the print has become opaque.

At a point after two days, the printed (and grown) pattern was removed from the support matrix, by placing the container with the print in a 10×PBS bath, which is being stirred. When the PBS contacts the hydrogel, the PBS reduces the viscosity of the hydrogel support matrix, allowing for the release of the printed pattern.

Complex patterns can be created in this manner, by printing a plurality of patterns using one or more inks. For example, in some embodiments, a first printed pattern is produced using a first ink composition. In some embodiments, a second printed pattern is then produced in the same hydrogel matrix using a second ink composition. The second ink may be different from the first ink. In some embodiments, a portion of the second pattern may be in contact with a portion of the first pattern. In some embodiments, a third printed pattern is then produced in the same hydrogel matrix using a third ink composition. The third ink may be different from the second ink and/or may be different from the first ink. In some embodiments, at least a portion of the third pattern may be in contact with a portion of the second pattern. In some embodiments, a portion of the third pattern may be in contact with a portion of the first pattern. In some embodiments, a portion of the third pattern may be in contact with a portion of the first pattern and a portion of the second pattern. In some embodiments, the pattern is cured after all inks are printed. In some embodiments, the pattern is cured separately after each ink is printed (e.g., the first pattern is cured after the first ink is printed, the second pattern is cured after the second ink is printed, etc.).

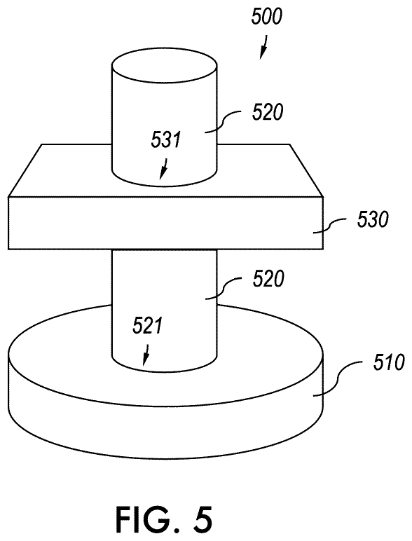

Such a complex pattern can be seen in reference to FIG. 5. In FIG. 5, a printed composite 500 can be seen. The first ink is used to print a first pattern 510 (here, the cylindrical base) using a first ink. The first ink may include a first pre-polymer composition and a first microorganism. A second pattern 520 (here, the long central pole extending upwards from the base) is then printed using a second ink, where a portion 521 of the second pattern is in contact with the first pattern. The second ink may include the first (or a second) pre-polymer composition, and may include the first (or a second) microorganism. For such composites, at least one characteristic of the second ink should be different from the first ink. After, a third pattern 530 (here, the square portion appearing about midway up the second pattern) is then printed using the first ink or a third ink. The third ink may include the first or second (or a third) pre-polymer composition, and may include the first or second (or a third) microorganism. For such composites, if a third ink is used, at least one characteristic of the third ink should be different from the first ink, and at least one characteristic should be different from the second ink. This process may be repeated until a complete composite has been printed. Preferably, a single curing step at the end of the printing is used. However, in some embodiments, multiple curing steps may be required, such as after each ink is printed, or after two or more inks have been printed but before all inks have been printed.

Those skilled in the art will recognize or be able to ascertain using no more than routine experimentation, many equivalents to the specific embodiments of the invention described herein. Such equivalents are intended to be encompassed by the following claims.

What is claimed:

1. A method for 3D printing engineered living materials, comprising:
providing an ink composition comprising a pre-polymer composition and a microorganism, the pre-polymer composition comprising a polymerizable monomer, a cross-linking agent, a photoinitiator, and a solvent;
3D printing a pattern in a hydrogel support matrix using the ink composition, the hydrogel support matrix being in a container; and
forming a 3D printed engineered living material by curing the 3D printed pattern.

2. The method according to claim 1, wherein the microorganism is a species of E. coli, A. adeninivorans, S. cerevisiae, C. glutamicum, or a combination thereof.

3. The method according to claim 1, wherein providing the ink composition comprises:
forming an ink composition by mixing a pellet having a known cell amount with a pre-polymer composition;
centrifuging the ink composition; and
loading the centrifuged ink composition into a container for injection.

4. The method according to claim 3, wherein the container for injection is a syringe.

5. The method according to claim 3, wherein the pellet is formed by:
inoculating cells of the microorganism into a growth medium;
growing the inoculated cells in the growth medium; and
centrifuging the grown cells into a pellet.

6. The method according to claim 1, wherein the polymerizable monomer is acrylamide.

7. The method according to claim 1, wherein the cross-linking agent is N,N′-methylenebis(acrylamide).

8. The method according to claim 1, wherein the photoinitiator is 2-Hydroxy-4′-(2-hydroxyethoxy)-2-methylpropiophenone.

9. The method according to claim 1, further comprising forming the pre-polymer composition by mixing the polymerizable monomer, the cross-linking agent, the photoinitiator, and the solvent.

10. The method according to claim 9, further comprising mixing a thickening agent into the pre-polymer composition.

11. The method according to claim 10, wherein the thickening agent is sodium alginate.

12. The method according to claim 1, further comprising forming a hydrogel support matrix by:
forming a hydrogel composition by mixing a sterile growth medium with a thickening agent;
eliminating bubbles by centrifuging the hydrogel composition; and
loading the hydrogel composition into a container for printing into.

13. The method according to claim 1, wherein curing the 3D printed pattern comprises irradiating the 3D printed pattern with at least one wavelength of light configured to activate the photoinitiator.

14. The method according to claim 1, further comprising reducing a viscosity of the hydrogel matrix to allow the 3D printed engineered living material to be released from the matrix.

15. The method according to claim 14, wherein reducing the viscosity of the hydrogel matrix comprises contacting at least part the hydrogel matrix with a buffered solution.

16. The method according to claim 15, wherein the buffered solution is 10× phosphate buffered solution.

17. A system for 3D printing engineered living materials, comprising:
a container comprising a hydrogel support matrix;
a reservoir containing an ink, the ink comprising a pre-polymer composition and a microorganism, the pre-polymer composition comprising a polymerizable monomer, a crosslinking agent, a photoinitiator, and a solvent;
a print head configured to deposit ink from the reservoir into the hydrogel support matrix;
a light source configured to irradiate the deposited ink with at least one wavelength of light capable of activating the photoinitiator; and
a processor configured to control the print head and light source.

18. The system according to claim 17, wherein the print head comprises an injection needle, the injection needle having a gauge between 15 and 34.
